(19) United States
(12) Patent Application Publication
(10) Pub. No.: US 2019/0256391 A1
(43) Pub. Date: Aug. 22, 2019

(54) CLARIFYING WATER AND WASTEWATER WITH FUNGAL TREATMENT/BIOFLOCCULATION

(71) Applicant: THE REGENTS OF THE UNIVERSITY OF CALIFORNIA, Oakland, CA (US)

(72) Inventors: Ruihong ZHANG, Davis, CA (US); Tyler BARZEE, Davis, CA (US)

(21) Appl. No.: 16/317,373

(22) PCT Filed: Jul. 17, 2017
(86) PCT No.: PCT/US2017/042453

§ 371 Date: Jan. 11, 2019

Related U.S. Application Data
(60) Provisional application No. 62/362,999, filed on Jul. 15, 2016.

(51) Int. Cl. C02F 3/34; C02F 3/28; C02F 1/54; C12N 1/14

(52) U.S. Cl. CPC C02F 3/347; C12N 1/14; C02F 1/54; C02F 3/28

(57) ABSTRACT

Anaerobic digestion is a widely used biotechnology for converting food, agricultural, and other organic wastes into biogas energy but produces nutrient-rich liquid effluent (digestate) that often requires costly disposal. Using digestate and similar wastewaters to produces microalgae for biodiesel or biochemical production can provide many economic and environmental benefits by offsetting fossil fuels. However, two aspects of microalgal production severely hinder the sustainability of the technique especially in arid regions: high energy use associated with the harvest of small microalgal cells and large volumes of water required to reduce concentrations of inhibitory compounds such as ammonia. We have compared the nutrient removal and pelletization potential of an easily harvested biofilm of robust and protective fungi evolved to high ammonia environments (ammonia fungi) with less resilient oleaginous microalgae for high strength wastewater treatment and biodiesel production. Preliminary calculations suggest that the ammonia fungi-algae pellets will require less dilution water and pH adjustment for growth in high-strength food waste digestate over the control fungi (Aspergillus sp.)-algae pellets. Impacts of pH on the surface charge (zeta potential) and pelletization of the fungi and microalgae will be compared among species and discussed in relation to impacts on pelletization potential.

### STATEMENT REGARDING FEDERALLY SPONSORED RESEARCH OR DEVELOPMENT

This invention was made with government support under Contract Number ARV-15-008, awarded by the California Energy Commission. The Government has certain rights in the invention.

### BACKGROUND OF THE INVENTION

Water used in various systems can accumulate undesirable content such as particulate matter, bacteria, algae, viruses, fungi, and pollutants. Examples of these water systems include cooling towers, evaporative coolers, swimming pools, fountains, sewage wastewater systems, water troughs for agricultural animals, agricultural runoff, and fisheries. If the undesirable content in these water systems is not treated, it can lead to broken devices, waterborne diseases, and other ill effects.

There are several existing options to treat water systems. For example, chlorination kills biological growth, desalination removes salt, and filtration removes particulate matter. A water system with undesirable content may bleed off water, and the water system is replenished with feed water that does not contain pollution, biological growth, etc. However, the use of chemicals or the constant replenishing of water can substantially increase costs associated with the maintenance of water quality.

Alternative water treatment options such as ultraviolet (UV) lamps can kill biological growth in water. However, UV lamps generally do not help with hyper-concentration and deposition of water-borne solids. Therefore, there is a need for a water treatment system that can function as a disinfectant and reduce the deposition of water-borne solids, while reducing the costs associated with such water treatment.

### BRIEF SUMMARY OF THE INVENTION

The present invention provides a solution to these and other shortcomings of present methods of clarifying an aqueous medium containing suspended solids. The present method provides a novel method of treating aqueous media, e.g., water and wastewater, to remove solids, e.g., microalgae, bacteria and other suspended solids using filamentous fungal bioflocculation, thereby achieving clarification of the aqueous medium. An exemplary method includes culturing filamentous fungi in a nutrient solution to form a filamentous fungi culture. The fungi thus cultured are added to the turbid water or wastewater to suspended solids, e.g., microalgae and bacteria cells. The pellets of filamentous fungi entrapping the suspended solids are then removed from the treated water or wastewater. Different fungi can be used for clarifying water and wastewater depending on the operating conditions of the device and/or method of the invention.

The present invention has many environmental and industrial applications. It can be used to treat natural water bodies, such as lakes and streams, or used to harvest algae and bacteria from industrial processes for biomass production. It can reduce energy costs associated with traditional methods of solid flocculation and removal for the purpose of water clarification and eliminate or minimize chemical addition requirements. This invention also has applications to microalgae production and wastewater treatment where it reduces the need for high-energy processes like autoclaving/pasteurization and centrifugation/microfiltration to harvest microalgal and bacterial cells.

Other methods of reducing turbidity in water/wastewater treatment and biomass production processes often require high energy operations such as centrifugation/microfiltration and/or sterilization/pasteurization. The present invention reduces energy costs for water clarification, e.g., through microfiltration, because the pellets of the complex between the filamentous fungus and the suspended solids are readily harvested using coarse filtration, a very low energy process. Additionally, the process does not require sterile water or pure cultures because the fungi pellets harvest both bacteria and microalgae.

Small cells (2-20 μm) and low cell density in solution (0.3-5 g/L) make efficient harvest of algae difficult. Moreover, biomass recovery can required up to 50% of the total energy cost. Thus, new methods and devices for separating algae from solutions are needed.

### BRIEF DESCRIPTION OF THE DRAWINGS

FIG. 1A. C. cinereus harvesting of bacteria and algae (Day 1 and Day 2).

FIG. 1B. C. cinereus harvesting of bacteria and algae (Day 3 and Day 4).

FIG. 2. N. crassa harvesting of bacteria and algae.

FIG. 3. Digestate ultrafiltration permeate composition

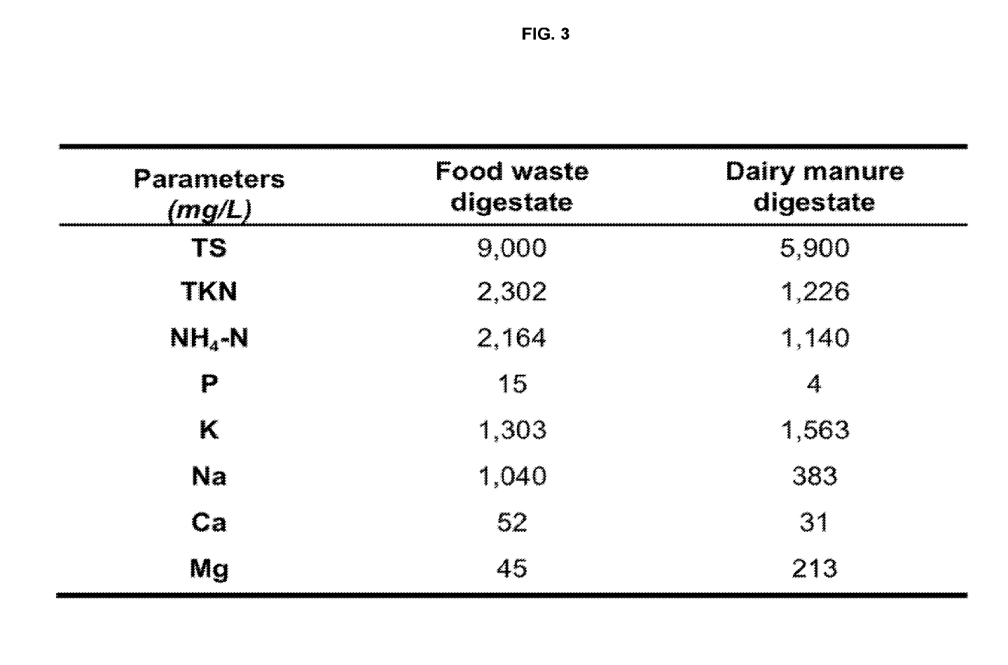

FIG. 4. Ammonia inhibition.

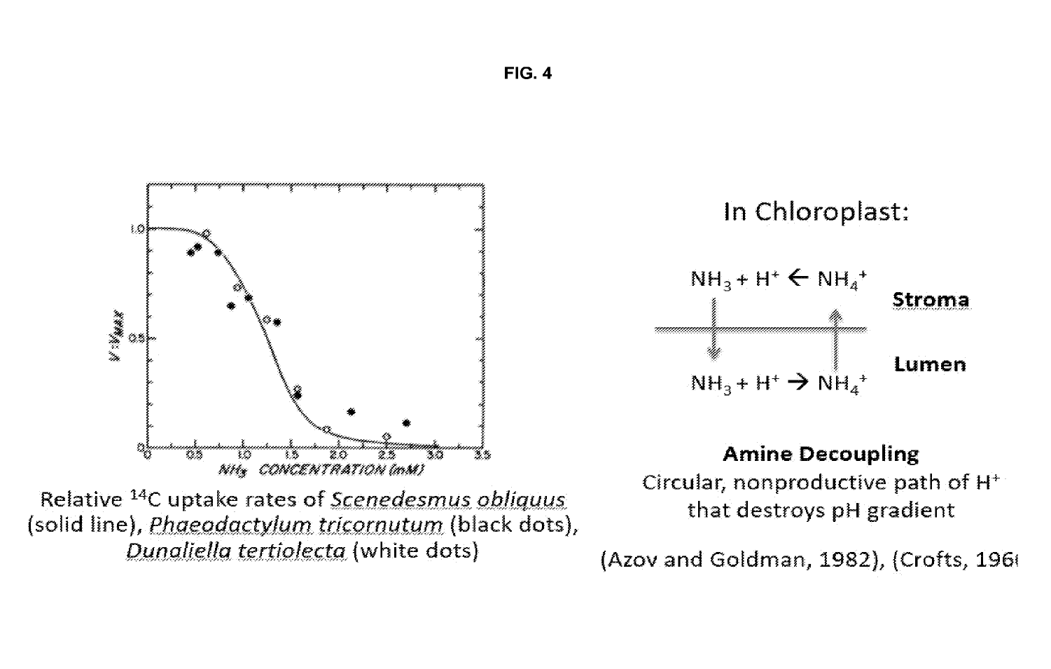

FIG. 5. Exemplary dilution scheme for pelletization.

FIG. 6. Ammonia removal by pellets in diluted food waste digestate permeate.

FIG. 7. Ammonia and COD removal.

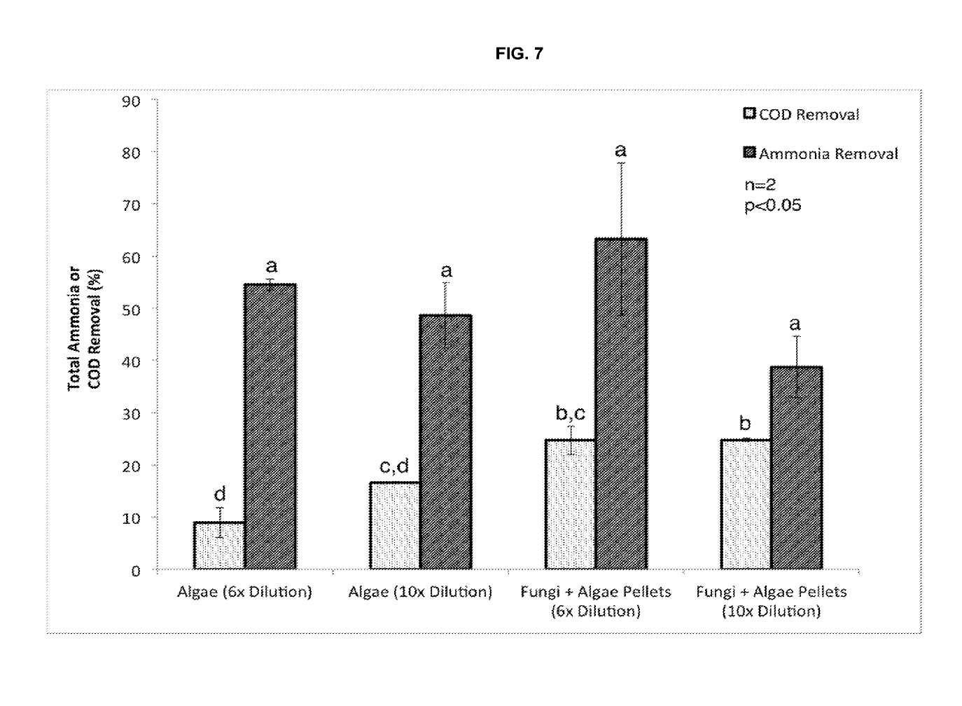

### DETAILED DESCRIPTION OF THE INVENTION

The present invention provides methods and devices for clarifying aqueous media containing one or more suspended solid. Exemplary methods of the invention include contacting an aqueous medium containing suspended solids with one or more filamentous fungus. The filamentous fungus promotes the flocculation of the suspended solid, facilitating it removal as a component of a complex, e.g., a pellet, with the filamentous fungus.

Also provided is a device of use for practicing the method of the invention. An exemplary device of the invention includes a first functional region in which the aqueous medium with the suspended solid is contacted with the filamentous fungus. The suspended solid may flocculate with the filamentous fungus in this region or the combination of filamentous fungus and suspended solid can be passed to a second region of the device in which the flocculation progresses or is completed. An exemplary device of the invention further provides a component for separating the complex between the filamentous fungus and the suspended solid for the remainder of the aqueous medium.

The filamentous fungal cell of use in the method and device of the invention may be any filamentous fungal cell. Filamentous fungi include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic.

In the present invention, the filamentous fungal cell may be a cell of a species of, but not limited to, Acremonium, Aspergillus, Fusarium, Humicola, Myceliophthora, Mucor, Neurospora, Penicillium, Thielavia, Tolypocladium, and Trichoderma or teleomorphs or synonyms thereof. Known teleomorphs of Aspergillus include Eurotium, Neosartorya, and Emericella. Strains of Aspergillus and teleomorphs thereof are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). Known teleomorphs of Fusarium of the section Discolor include Gibberella gordonii, Gibberella cyanea, Gubberella pulicaris, and Gibberella zeae.

In an exemplary embodiment, the filamentous fungal cell is an Aspergillus cell. In another exemplary embodiment, the filamentous fungal cell is an Acremonium cell. In another exemplary embodiment, the filamentous fungal cell is a Fusarium cell, e.g., a Fusarium cell of the section Elegans or of the section Discolor. In another exemplary embodiment, the filamentous fungal cell is a Humicola cell. In another exemplary embodiment, the filamentous fungal cell is a Myceliophthora cell. In another exemplary embodiment, the filamentous fungal cell is a Mucor cell. In another exemplary embodiment, the filamentous fungal cell is a Neurospora cell. In another exemplary embodiment, the filamentous fungal cell is a Penicillium cell. In another exemplary embodiment, the filamentous fungal cell is a Thielavia cell. In another exemplary embodiment, the filamentous fungal cell is a Tolypocladium cell. In another exemplary embodiment, the filamentous fungal cell is a Trichoderma cell. In another exemplary embodiment, the filamentous fungal cell is an Aspergillus oryzae, Aspergillus niger, Aspergillus foetidus, Aspergillus nidulans, or Aspergillus japonicus cell. In another exemplary embodiment, the filamentous fungal cell is a Fusarium strain of the section Discolor (also known as section Fusarium). For example, the filamentous fungal cell may be a Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecioides cell. In another prefered embodiment, the filamentous parent cell is a Fusarium strain of the section Elegans, e.g., Fusarium oxysporum. In another exemplary embodiment, the filamentous fungal cell is a Humicola insolens or Humicola lanuginosa cell. In another exemplary embodiment, the filamentous fungal cell is a Myceliophthora thermophilum cell. In another exemplary embodiment, the filamentous fungal cell is a Mucor miehei cell. In another exemplary embodiment, the filamentous fungal cell is a Neurospora crassa cell. In another exemplary embodiment, the filamentous fungal cell is a Penicillium purpurogenum cell. In another exemplary embodiment, the filamentous fungal cell is a Thielavia terrestris cell. In another exemplary embodiment, the Trichoderma cell is a Trichoderma reesei, Trichoderma viride, Trichoderma longibrachiatum, Trichoderma harzianum, or Trichoderma koningii cell.

As used herein, the term “wastewater” refers to any stream of water containing an undesirable contaminant including byproducts of environmental, industrial, and municipal processes. In addition, the term “wastewater” encompasses a contaminated stream of water suited for treatment to produce potable water or drinking water.

The effectiveness of the device and method of the instant invention is readily assessed by art standard methods. See, for example, U.S. Patent Publication No. 20140131259. In one embodiment, a method of determining the turbidity of wastewater includes receiving a signal indicative of an amount of light scattered by the wastewater and sampling the signal to produce a plurality of signal sample values. These sample values are compared to a threshold, and the sample values falling inside the threshold identified. The method further includes determining the turbidity of the wastewater based on the sample values falling inside the threshold.

In some embodiments of the invention, the signal indicative of the amount of light scattered by the wastewater may be generated by detecting an amount of light scattered from a beam of light by the wastewater, in which case the signal may have a higher value (i.e., more light would be detected) for turbid water than for clear water. In other embodiments, this signal may be generated by detecting an amount of light transmitted through the wastewater, in which case the signal may have a lower value (i.e., less light would be detected) for turbid water than for clear water.

The devices of the invention, incorporating the filamentous fungi are designed according to art-accepted standards, and provide for a region in which the filamentous fungi contact the wastewater, thereby resulting in bioflocculation of at least a portion of the solid contaminants in the wastewater. Removal of the solid contaminants results in a measureable reduction in wastewater turbidity and a measureable improvement in water clarity. Exemplary devices are analogous to those art-recognized devices utilizing flocculation and settling to reduce turbidity and improve clarity of wastewater. An exemplary device and method is set forth in U.S. Patent Application Publication No. 20140209523.

The present invention is also of use in producing products of value from algal or bacterial cells, including biofuel, and commodity and specialty chemicals. In exemplary embodiments, the cells are removed by the device and/or method of the invention from a fermentation broth after the desired product is produced by the cells. In another embodiment, the cells are entrained and immobilized by the filamentous fungus during the synthesis of the desired product.

Anaerobic digestion is a widely used biotechnology for converting food, agricultural, and other organic wastes into biogas energy but produces nutrient-rich liquid effluent (digestate) that often requires costly disposal. Using digestate and similar wastewaters to produce microalgae for biodiesel or biochemical production can provide many economic and environmental benefits by offsetting fossil fuels. However, two aspects of microalgal production severely hinder the sustainability of the technique especially in arid regions: high energy use associated with the harvest of small microalgal cells and large volumes of water required to reduce concentrations of inhibitory compounds such as ammonia. We have compared the nutrient removal and pelletization potential of an easily harvested biofilm of robust and protective fungi evolved to high ammonia environments (ammonia fungi) with less resilient oleaginous microalgae for high strength wastewater treatment and biodiesel production. Preliminary calculations suggest that the ammonia fungi-algae pellets will require less dilution water and pH adjustment for growth in high-strength food waste digestate over the control fungi (Aspergillus sp.)-algae pellets. Impacts of pH on the surface charge (zeta potential) and pelletization of the fungi and microalgae will be compared among species and discussed in relation to impacts on pelletization potential.

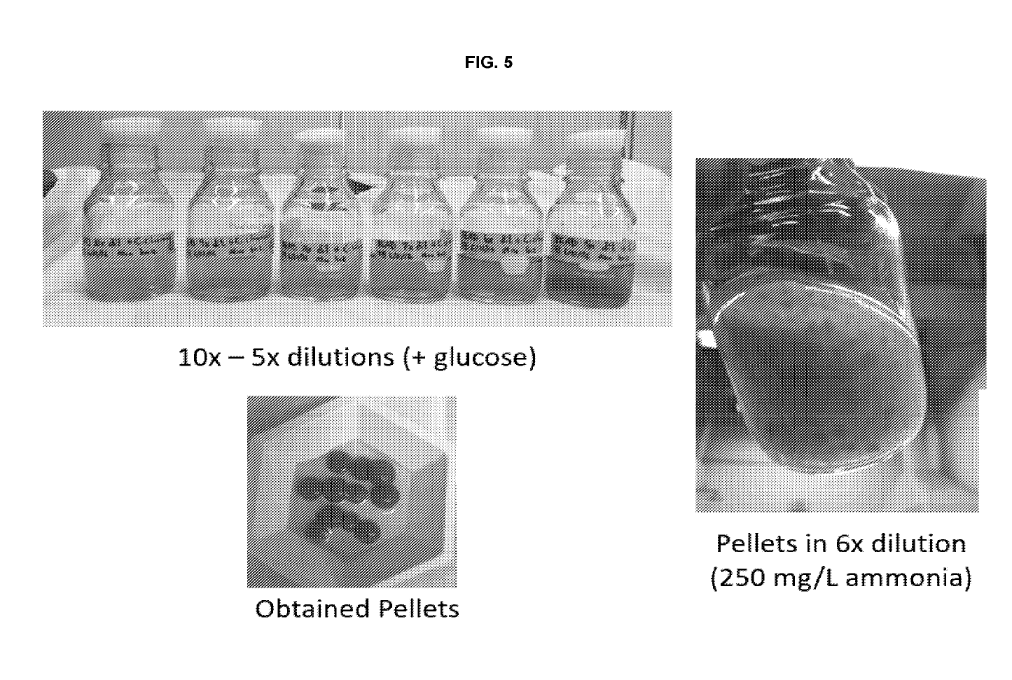

Digestate has high ammonia content. For example, food waste digestate [NH3]=85 mg/L=5 mM. Substrate inhibition necessitates a 10-20× dilution. FIG. 5. Ammonia fungi are an ecologically important class found in forests responding to high N-loading events (defcation, decay, urination). They thrive in the high ammonia environment found in digestate. Though these fungi have been studied ecologically, the have found minimal application in biotechnology, and in wastewater treatment.

In an exemplary embodiment, the present invention provides a method of clarifying digestate and/or digestate permeate using an ammonia fungi, e.g., Coprinopsis cinerea. FIG. 5. Also provided is a device utilizing an ammonia fungi to provide this clarification.

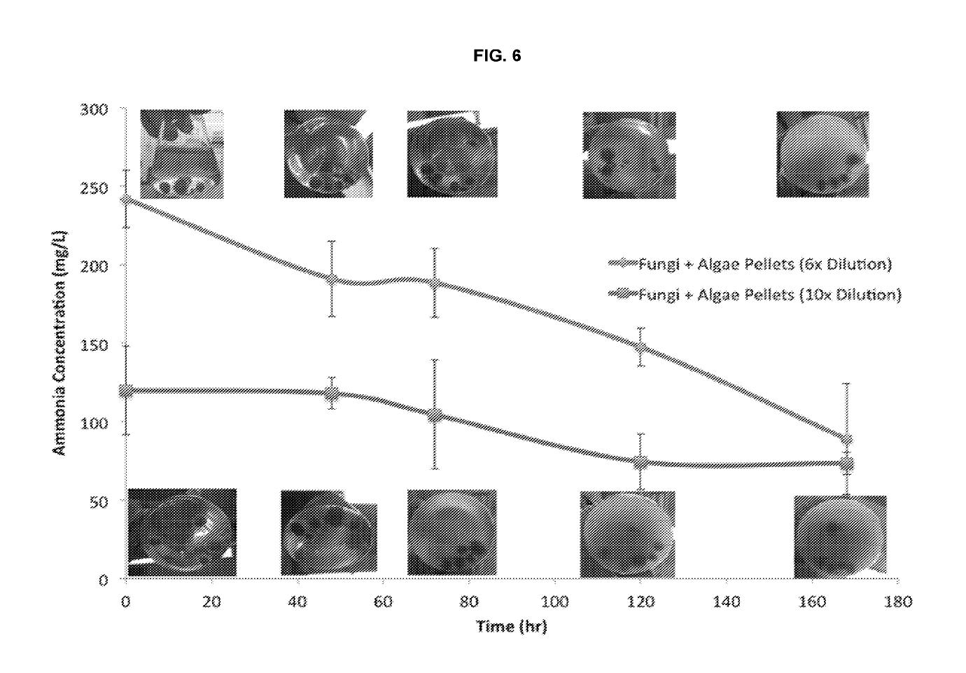

In an exemplary embodiment, the method and/or device of the invention are of use to reduce the ammonia content of a solution, such as a digestate. FIG. 6.

In an exemplary embodiment, the method and device of the invention are practiced as a component of an integrated algae production scheme. In various embodiments, the method and/or device of the invention is a component of an integrated algae production using anaerobic digester effluent. In a still further exemplary embodiment, the integrated algae production scheme further includes infrared drying.

In various embodiments, the method and/or device of the invention is of use to clarify a digestate from an anaerobic digestion process.

The Examples set forth below are provided to illustrate an exemplary embodiment of the invention and are to be interpreted as limiting the invention.

### EXAMPLES

The following shows examples of invention:

### Example 1

Fungi Coprinopsis cinerea and Neurospora crassa were grown on PDA or other suitable solid media such as PD-rabbit dung agar. The plate is incubated at 25-30° C. for two to three days until white, puffy mycelium is formed with black spore tips (C. cinereus) or orange mycelium with fluffy conidia (N. crassa) were established. The surface was either washed with sterilized water or a small piece (1 mm²) of growth is removed and transferred to a water solution. The solution is vortexed for thirty seconds. The larger particles are allowed to settle and the supernatant is transferred to another small tube. The spore suspension was then observed with a hemocytometer and the spore number is counted.

The spores were inoculated into BG-11 cyanobacteria growth media and shaken at 150-250 RPM at 20-30° C. for three days until pellets form.

Microalgae was grown in unsterile conditions in BG-11 media or 10× diluted anaerobic digestate permeate, which is the liquid fraction of digestate. Dilution was made with water or BG-11 media. Both algae and bacteria were noticeable in the liquid.

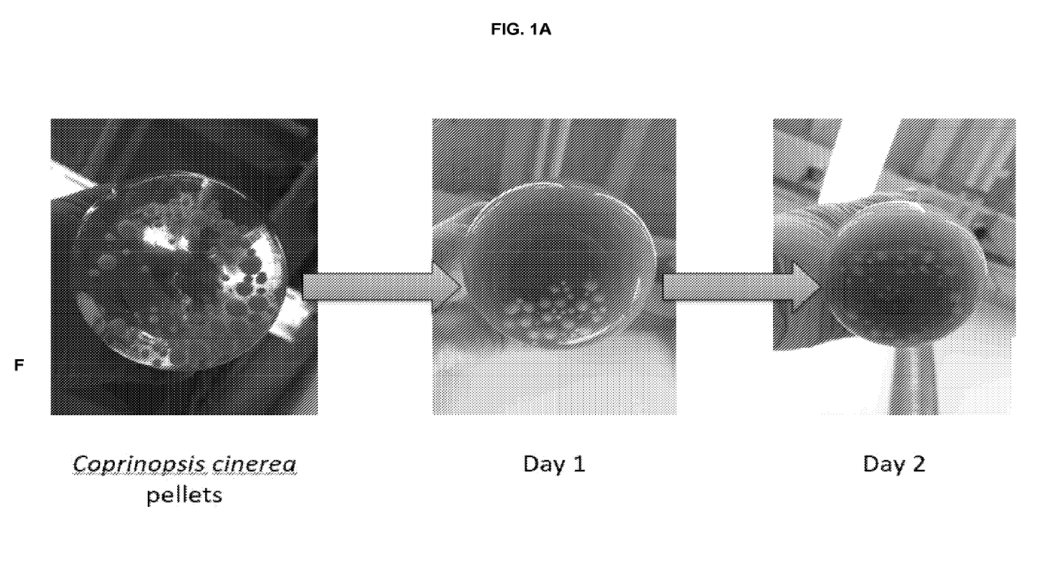

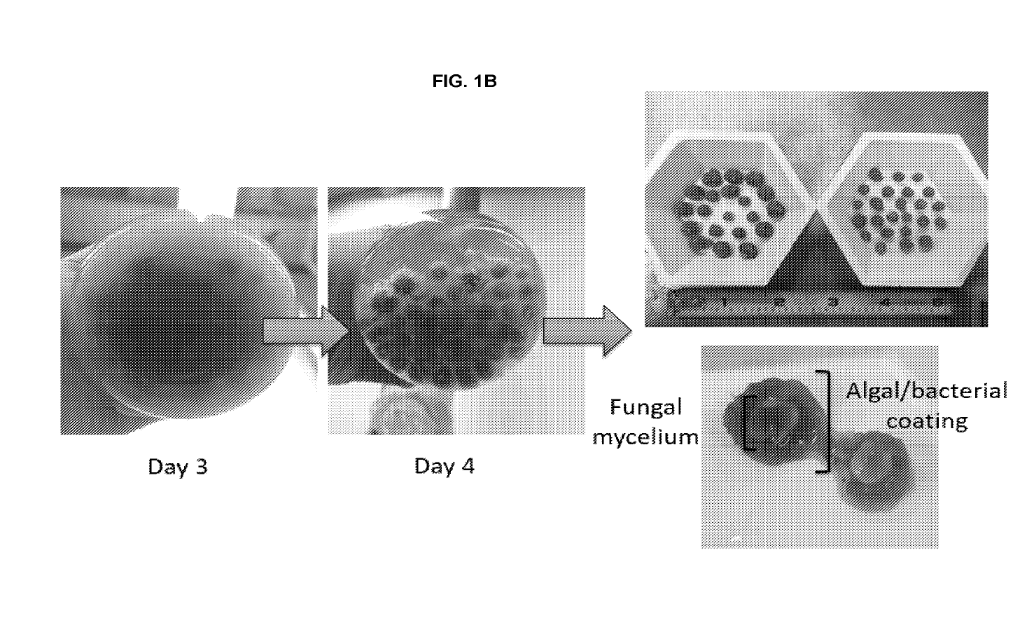

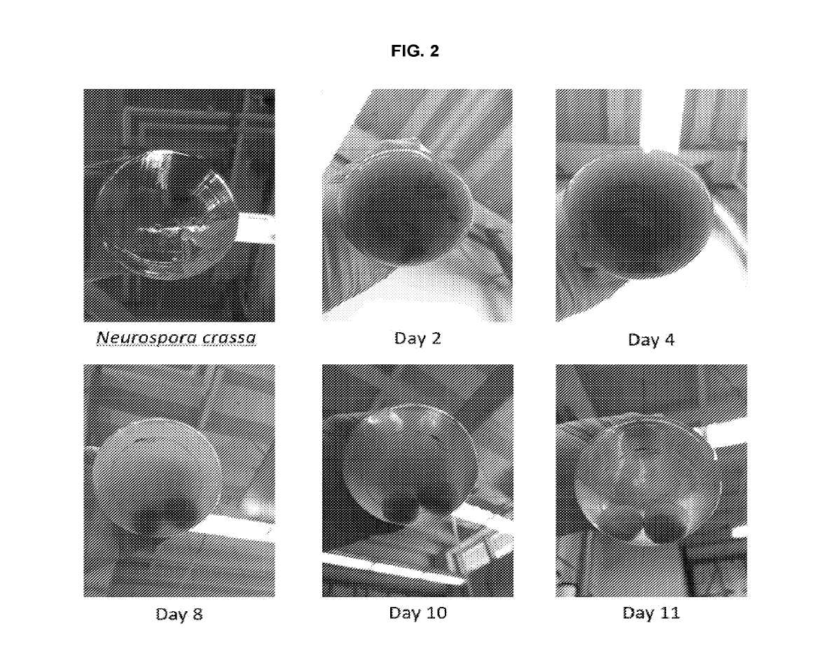

The fungi pellets were added to the algae and bacteria containing liquid. The flask was then shaken at 150-250 RPM at 20-30° C. for four days. Fungal pellets were trapped. Removed algae and bacterial cells and clarified water are shown in FIG. 1 and FIG. 2. Then the pellets were removed from water by coarse filtration (FIG. 1 and FIG. 2).

It was shown that Coprinopsis cinerea works well at neutral pH (6-8) and N. crassa works well at low pH (4-5).

### Example 2

### Spore Production

Fungal spores can be produced on a solid substrate in a process called “Solid State Fermentation”. Industry solid state fermentations often consist of polypropylene bag, column reactors, or tray reactors. Polypropylene bag reactors consist of loading substrate into a bag, inoculating with a spore solution or fungal mycelium. Bag reactors are often limited by gas exchange. Column reactors consist of a cylindrical column filled with substrate where air can be pumped from one end to the other. Column reactors offer advantages in heat and volatile compound removal and favorable gas exchange to the fungi. Tray reactors consist of stacks of trays filled with substrate and inoculated with fungi. The environment around the trays is ventilated to remove heat and volatile compounds and has potential to supply a large surface area with oxygen.

In this invention, a column reactor is used. The other types of reactors could also be used but have not been tested yet. The column is filled with a cheap substrate such as cracked corn and is maintained at a moisture content of 30-40% to encourage fungal growth. The substrate could also consist of other grains such as rice, barley, or wheat, or agricultural residues like wheat straw or corn stover. Depending on the species of fungi, pretreatment of lignocellulosic materials may be necessary because required enzymes may be lacked in certain fungal species. The column is inoculated with a fungal spore suspension by soaking the corn in a spore suspension isolated from a pure fungi culture. Ideally, the growth substrate is autoclaved prior to inoculation to ensure that only the target fungi grows. Autoclaving may also aid in the degradation of the substrate by the fungi. The substrate is incubated at 30 deg C with or without aeration for 6 days until a suitable amount of fungal biomass is created and spores are clearly present.

The spores can then harvested by adding the biomass to a vibratory screen with a sieve of a suitable size to allow spore separation from the rest of the substrate. The dry spores can then be added to a small volume of water to create a highly concentrated spore suspension stock for inoculation of more cracked corn or of media for the purposes of producing fungal pellets. Alternatively, the dry biomass can be washed with sterile water in the vibratory screen to create a spore suspension.

### Pellet Production

Fungal pellets are produced from the spore suspension created from solid state fermentation of a suitable substrate. The spore suspension contains a concentration of spores ≥1*10⁶ spores/mL. Spores are quantified by diluting the suspension and counting with a hemacytometer. The spore suspension can also be diluted to different levels and measured with a spectrophotomer to create a calibration curve between spore number and absorbance at a specific wavelength.

The spore suspension is inoculated to a sterile media containing nutrients and sugars sufficient for the growth of fungi. Slightly acidic conditions may favor fungal growth so basic or neutral media can be adjusted to pH of 3-4 with citric acid or another suitable acid if desired. Depending on the species of fungi, acidification may not be necessary. The spores should be inoculated in a volume to create a concentration in the media of 1*10⁴ spores/mL. The solution must then be agitated to favor the formation of fungal pellets instead of one mass of fungal mycelium. Agitation can be carried out by stirring with an impeller, shaking with a rotating stage, aerating, or using a combination of physical agitation and aeration. Shaking at higher rotational speeds favors the production of smaller pellets. Generally, a speed of 250 rpm produces pellets less than 0.5 inches in diameter. Aeration provides benefits of agitating while simultaneously delivering oxygen to the medium. In lab scale experiments, aeration levels of 1-2 vessel volumes per minute were required to produce pellets. Higher aeration rates favor the production of smaller pellets to a point but seems to level off above 2 vessel volumes per minute. Over aeration may disrupt pellet formation and can expose the fungi to oxidative stress. The aeration tube must be placed in a location that induces movement of the whole body of liquid, especially the bottom since fungal pellets tend to sink. If the whole column of liquid is not moving, the fungi may tend to gather in one location and form large aggregates instead of many individual pellets that are needed to provide large surface area for algae/bacteria harvesting in later stages. Simultaneous aeration and physical agitation from impellers or shakers may provide benefits but has not yet been tested. Fungal pellet production usually occurs within 30-48 hours after inoculation but depends on the growth characteristics of the fungi, reactor conditions, and the nutrient content of the media.

### Bacteria and Algae Harvesting

The fungal pellets produced are then isolated with a coarse separation step using a bag filter with a synthetic membrane with a large pore size or other cheap material such as cheesecloth. The pellets are then added, in non-sterile conditions, to a solution containing microalgae and bacteria. In certain cases, it may be beneficial to heat treat the solution by heating to 60-75 deg C and/or adding glucose to the solution to encourage fungal growth. However, it is generally not necessary to do either step. Experiments have been conducted adding fungi to an algae solution on a dry mass basis of 0-3 gram of fungi volatile solids per gram of algae volatile suspended solids (0-3 g fungi/g algae). From the experiment, it appeared that algae harvesting potential plateaued around a fungi loading of 1.5 g fungi/g algae but 3 g/g loading resulted in much faster algae harvesting. A 3 g/g fungi loading resulted in desirable algae/bacteria harvesting in less than 24 hours while lower loading rates could take upwards of 3 days to reach the same harvesting level. Once the fungi pellets are added to the solution, the reactor may be agitated and/or aerated with similar procedures as the pellet production step. Optimization studies have not been carried out yet on this step. Similarly to the separation of fungal pellets from their medium, a coarse separation step can then be undertaken to separate the fungi-algae-bacteria pellets from the liquid.

### REFERENCES

1) “Microalgae culture and harvest”. WO 2013055887 A1. Bo Hu, Rongsheng R. RUAN, Jianguo Zhang, Wenguang Zhou.
   https://www.google.com/patents/WO2013055887A1?cl=en&dq=fungi+algae+harvest&h1=en&sa=X&ved=0ahUKEwjRi7zHxvPNAhVS_WMKHfw1DtoQ6AEIHjAA
2) “System and Method of Co-Cultivating Microalgae with Fungus”. US 20120282651 A1. Joshua (Shuhua) YUAN. Shangxian XIE.
   https://www.google.com/patents/US20120282651?dq=fungi+algae+harvest&h1=en&sa=X&ved=0ahUKEwjRi7zHxvPNAhVS_WMKHfw1DtoQ6AEILjAC
1) Zhang, J, & Hu, B. (2012) A novel method to harvest microalgae via co-culture of filamentous fungi to form cell pellets. Bioresource Technology, 114, 529-535.
2) Zhou, W, Cheng. Y , Li, Y , Wan, Y., Liu. Y , Lin. X, & Ruan. R. (2012). Novel fungal pelleti/ation-assisted technology for algae harvesting and wastewater treatmen. Applied biochemistry and biotechnology, 167(2), 214-228.
3) Muradov. N., Taha, M., Miranda. A. F., Wrede. D . Kadali. K., Gujar. A., . . . & Mouradov, A. (2015). Fungal-assisted algal flocculation: application in wastewater treatment and bioluel production. Biotechnology for biofuels, 8(1), 1.

1. A device for clarifying wastewater having suspended solids, said device comprising at least a first region wherein said wastewater contacts at least one filamentous fungi, thereby flocculating at least a portion of said suspended solids and clarifying said wastewater.

2. The device according to claim 1, wherein said suspended solid is selected from algae (e.g., microalgae), bacteria and a combination thereof.

3. A method for clarifying wastewater having suspended solids, said method comprising contacting said wastewater with at least one filamentous fungus, thereby flocculating at least a portion of said suspended solids and clarifying said wastewater.

4. The method of claim 3, further comprising incubating the filamentous fungi under conditions in which the filamentous fungi grow.

5. The method according to claim 3, wherein spores of said filamentous fungus are produced by solid state fermentation of a substrate.

6. The method according to claim 5, wherein the substrate is a grain or a bran (e.g., rice bran or wheat bran).

7. The method according to claim 6, wherein the grain is cracked corn.

8. The method according to claim 5, wherein the spores of said filamentous fungus are collected from the substrate using a combination of vibrating screens and washing.

9. The method according to claim 5, wherein the filamentous fungus is collected from the substrate while the fungus is viable (i.e., not dead and/or dying).

10. The method of claim 3, further comprising separating said filamentous fungus and the floculated suspended solids from the wastewater.

11. The method according to claim 5, further comprising aerating and/or agitating the vessel in which the substrate and the at least one filamentous fungus are contained.

12. A method of isolating a desired compound from an aqueous suspension of cells synthesizing said compound, said method comprising entraining said cells in a filamentous fungus.

13. The method according to claim 12, wherein spores of said filamentous fungus are produced by solid state fermentation of a substrate.

14. The method according to claim 12, further comprising incubating the filamentous fungus under conditions in which the fungus grows.

15. The method of claim 13, wherein the substrate is a grain or a bran (e.g., rice bran or wheat bran).

16. The method of claim 15, wherein the grain is cracked corn.

17. The method according to claim 13, wherein the spores of said filamentous fungus are collected from the substrate using a combination of vibrating screens and washing.

18. The method according to claim 13, wherein the filamentous fungus is collected from the substrate while the fungus is viable (i.e., not dead and/or dying).

19. The method of claim 12, further comprising separating the filamentous fungus and the entrained cells from said compound.

20. The method according to claim 12, further comprising aerating and/or agitating the vessel in which the substrate and the at least one filamentous fungus are contained.
